(19) United States
(12) Patent Application Publication
(10) Pub. No.: US 2026/0199583 A1
(43) Pub. Date: Jul. 16, 2026

(54) SIZE-CLASSIFIER OF ADIPOCYTE CELLS AND LIPOSUCTION SYSTEM COMPRISING SAME

(71) Applicant: EUROMI, Andrimont (BE)

(72) Inventors: David Leleu, Andrimont (BE); Dimitri Lemaire, Andrimont (BE)

(73) Assignee: EUROMI, Andrimont (BE)

(21) Appl. No.: 19/133,852

(22) PCT Filed: Nov. 30, 2022
(86) PCT No.: PCT/EP2022/083780

§ 371 Date: May 29, 2025

(51) Int. Cl. A61M 1/00; C12M 1/00

(52) U.S. Cl. CPC A61M 1/893; C12M 47/04; A61M 2202/08; A61M 2205/7545

(57) ABSTRACT

A size-classifier (12) for dividing adipocyte cells into specific size fractions is provided inclusive of a carousel (14) rotatably mounted in a classifier vessel (12v) to rotate about a classifier axis (Z12). The carousel (24) is divided into compartments (14.1-14.n) separated by compartment walls, each having a transverse wall portion which is transverse to a radial direction of the carousel wall (14w). The transverse wall portion separating an i^{th} compartment (14.i) from a (i+1)^{th} compartment (14.(i+1)), with i=1 to (n−1), includes a mesh of i^{th} opening (Ri), smaller than an (i−1)^{th} opening (R(i−1)) of a transverse wall portion separating a (i−1)^{th} compartment (14.(i−1)) from the i^{th} compartment (14.i), (i.e., R(n−1)<R(n−2) . . . <Ri< . . . <R1). A liposuction kit-of-parts and assembly for liposuction and treatment of the liposuction aspirate as to size classification is also provided.

### TECHNICAL FIELD

The present invention concerns a size-classifier for dividing adipocyte cells collected from a liposuction aspirate into specific size fractions. The invention also concerns a kit of parts and an assembly for an apparatus for liposuction of adipose tissue comprising the size-classifier. The kit of parts or assembly comprising the size classifier of the present invention are configured for extracting adipose tissue from a location of a patient and cleaning and isolating into fractions of selected size ranges ready or nearly ready for further use, such as further treatment or re-injecting adipose cells from said adipose tissue into another location of said patient (or of another patient). In particular, the kit of parts or assembly comprise (A) a liposuction device configured for extracting the adipose tissue, and (B) the size-classifier in fluid communication with and downstream of the liposuction device. Preferably, one or both of a separation unit and a fluidization unit are provided in fluid communication with both liposuction device and size-classifier and located between the two in this sequence.

The size classifier of the present invention allows adipose cells from liposuction aspirates to be divided into specific size ranges optimized for topical applications including re-injection applications or other further treatments. The assembly ensures sterile conditions of the adipose cells from the extraction locus to the re-injection locus.

### BACKGROUND OF THE INVENTION

“Adipose tissue” or (body) “fat” is loose connective tissue composed mostly of adipocytes or “fat cells”. Liposuction is the removal of adipose tissues from a location of a body. It can be applied for therapeutic reasons to treat obesity, which is an excess of adipose tissue, or it can be applied for cosmetic reasons to improve one's figure. It can also be used for collecting cells for autologous fat grafting in reconstructive surgery. In liposuction, a hollow cannula comprising an opening at or close to its free end, is inserted into the region of the body to be treated through a small incision in the skin. Adipose cells and other bodies including fibrous connective tissues resident in the adipose deposits and fluids such as blood, oil, and tumescence fluids, are aspirated through the lumen of the cannula which is connected to a vacuum source and thus driven into a container. Examples of liposuction devices are described e.g., in WO9844966, U.S. Pat. No. 4,536,180, WO2011146924, U.S. Pat. No. 5,911,700.

Adipose cells comprise numerous cells including unilocularwhite adipocytes, multilocular brown adipocytes which together represent the parenchyma, as well as other cells constituting the stroma-vascular fraction of adipose tissue including fibroblasts, macrophages, blood and endothelial cells, and mesenchymal stem cells. Unilocular white adipocytes make up about 95% of adipose tissue and are cells generally between 100 and 150 μm in diameter. Their cytoplasm is entirely occupied by triglycerides while their nucleus is located peripherally against the membrane. White adipocytes secrete a large number of peptides (“adipokynes”) such as resistin, the appetite and satiety regulating hormone leptin (anorectic hormone) and apelin. White adipocytes are responsible for the synthesis, storage and release of lipids.

Multilocular brown adipocytes are cells of the order of 30 μm in diameter. They contain numerous lipid droplets. They are the hibernating cells that participate in thermogenesis which is accomplished by fatty acid phosphorylation in the numerous cytochrome rich mitochondria, giving the cell its brown colour. These cells are connected to β-adrenergic nerve endings. The presence of brown adipocytes in adult humans has been demonstrated since 2009

Stromal vascular fraction (SVF) of cells includes preadipocytes, fibroblasts, vascular endothelial cells and a variety of immune cells which are present in the liposuction aspirates. Far from being inert, it has been established that adipose tissue can produce hormones and is an abundant source of CD34+ cells. CD34+ cells are a mixture of stem cells, progenitors, and white blood cells of various degrees of maturity.

When in the past the liposuction aspirates used to be disposed of after collection, more applications are being developed to re-use the precious components forming the adipose tissues thus aspirated. For example, in reconstructive surgery autologous fat cells can be grafted. In aesthetic surgery, improving one's figure is not restricted to removing adipose cells from locations where it is considered they are present in excess, but may also comprise giving volume to locations of the body considered as volume deficient. It has rapidly been proposed to re-inject a fraction of the adipose tissue extracted from one location of the body in excess of body fat, into another location deficient in adipose tissue, like e.g., lips, cheeks, breast. This solution is appealing since there is no risk of rejection of its own cells by the patient. The extraction of adipose tissue is often referred to as “liposuction”, whilst the re-injection of adipose cells is referred to as “lipofilling.” In cosmetic applications, a liposuction followed by a lipofilling is often artistically referred to as “liposculpture.”

Before being re-used in any application, the adipose tissues must of course first be separated from the undesirable other bodies. This is achieved with filters, which retain the solid adipose cells and some other solid foreign bodies and removes the liquids and small debris as filtrate. Examples of such filters are described e.g., in U.S. Pat. No. 8,858,518 and EP3596196.

Re-injection or re-use of a fraction of the adipose tissue extracted by liposuction is drawing more and more attention in therapeutical applications, in particular for the recovery of the stromal vascular fraction (SVF) of cells discussed supra, which have great value in medicine. As discussed supra, different components of the adipose tissues forming the liposuction aspirate have very different size ranges. Fractions of specific components of interest can therefore be separated based on their size.

WO2011146924 describes a system for extracting fat cells through a cannula subjected to ultrasonic vibration. The fat cells collected in a container can be filtered to separate the larger fat cells from the rest of the sample. Several successive filters of sizes between 200 and 800 μm can be arranged in series to optimise the separation process. When extracting fat tissue by liposuction, including ultrasonic, fat cells, and in particular white and brown adipocytes, are often extracted as large agglomerates of various cells, which are often damaged after extraction, causing necrosis of many cells.

CN207659440U, US2018/0037866, and BE1024139 describe size-classifiers for separating adipose cells by size fractions. The size-classifiers comprise a housing provided with filters of different sizes arranged in a sequence in series. The driving force for driving a flow of the adipose tissues through the various filters is vacuum applied by a vacuum pump, generally the same used for driving the aspiration of the adipose tissues through the cannula. The driving force for filtering is therefore limited to the power of the vacuum pump and is anyway limited to −1 bar.

It has been observed that with prolonged treatment of the adipose tissues under constant vacuum for driving the flow thereof through the various units for separating; cleaning; fluidizing, and isolating specific adipose cells for liposculpture or other re-use applications of adipose cells, these tended to dry and become more difficult to handle. Adding a saline solution (or physiological serum) to the adipose cells improved the situation. With vacuum being applied constantly, however, adding any fluid to the adipose tissues is a challenge.

There therefore remains a need in the art for an apparatus allowing an accurate size classification of pre-cleaned adipose tissues and, at the same time, allowing adding fluid thereto to maintain substantially constant the moisture content of the adipose tissues during their treatment operations to preserve their integrity and facilitate their handling and flowing.

The present invention proposes a size-classifier for use in an assembly for aspirating adipose tissues, treating them and separating the adipose cells by size fractions with high accuracy and reproducibility and in a sterile environment, prior to their re-use in the same or a different patient or prior to further treatment. This and other advantages of the present invention are presented in continuation.

### SUMMARY OF THE INVENTION

The present invention is defined in the appended independent claims. Preferred embodiments are defined in the dependent claims. In particular, the present invention concerns a kit of parts for a size-classifier for dividing adipocyte cells into specific size fractions. The size-classifier comprises a classifier vessel, a carousel, a classifier inlet opening and a classifier outlet opening, and a driving coupling element.

The classifier vessel has a classifier floor, a classifier wall having a geometry of revolution about a classifier axis (Z12) and an open end closed by a classifier lid and defining a classifier inner volume. The carousel is rotatably mounted in the classifier inner volume to rotate about the classifier axis (Z12), and comprises,
a carousel vessel having a carousel floor, a carousel wall which has a geometry of revolution about the classifier axis (Z12) and an open end preferably sealed by a membrane, defining a carousel inner volume, wherein
the carousel inner volume is divided into a plurality of compartments separated from one another by compartment walls, each compartment wall comprising a transverse wall portion which is transverse to a radial direction of the carousel wall (14w),

The transverse wall portion separating a first compartment from a second compartment comprises a mesh of first opening (R1). The transverse wall portion separating an i^{th} compartment from a (i+1)^{th} compartment)), with i=2 to (n−1), comprises a mesh of i^{th} opening (Ri), smaller than an (i−1)^{th} opening (R(i−1)) of a transverse wall portion separating a (i−1)^{th} compartment from the i^{th} compartment, (i.e., R(n−1)<R(n−2) . . . <Ri< . . . <R1). The carousel floor of the n^{th} compartment comprises an opening in fluid communication with the classifier floor and with the outlet opening.

The classifier inlet opening is provided in the classifier lid in registration with the carousel and configured for giving a needle direct access to the compartments, through the optional membrane. The classifier outlet opening is provided in the classifier floor and is configured for evacuating liquids. The driving coupling element is configured for driving a rotation of the carousel relative to the classifier vessel when coupled to a motor.

The first opening (R1) can have a first diameter comprised between 200 and 1000 μm, preferably between 400 and 800 μm, more preferably between 500 and 700 μm. The (n−1)^{th} opening (R(n−1)) can have a (n−1)^{th} diameter comprised between 10 and 200 μm, preferably between 20 and 100 μm, more preferably between 40 and 80 μm.

The compartment walls can have different geometries. For example, the compartment walls can extend substantially radially from a central hub and change orientation to reach the carousel wall transverse to the radial direction of the carousel wall, forming with the radial direction an angle (α) preferably comprised between 45 and 120°, more preferably between 6° and 120°. The change of direction can form a corner, defining a segmented compartment wall or can be smooth defining a curved compartment wall. Alternatively, the compartment walls can be cylindrical walls coaxial with the classifier axis (Z12) and of different radii, defining annular compartments.

The opening in the carousel floor of the n^{th} compartment (14f.n) can be formed by,
no floor, or
a grid or mesh of openings larger than or equal to the (n−1)^{th} openings (R(n−1)) of the transverse wall portion of the (n−1)^{th} compartment wall, or
an aperture in the floor.

The driving coupling element can comprise a pinion of a rack-and-pinion mechanism, wherein the pinion is configured for being coupled to a rotation transmission cable to a motor. Alternatively, the driving coupling element can comprise magnets forming an electric motor whose stator is formed by the classifier wall and the rotor by the carousel wall.

The present invention also concerns a kit-of-parts for an apparatus for liposuction of adipose tissue. The kit-of-parts of the present invention comprises a liposuction device, a transfer tube, a source of vacuum, and a size-classifier as defined supra. The liposuction device has a cannula which is substantially linear, hollow, and elongated, comprising an inner lumen extending along a cannula axis (X2) from an inlet end (2i), provided with one or several openings for drawing adipose tissue into said lumen, to an outlet end located at an opposite end of the cannula. The liposuction device also comprises a handpiece comprising a cannula coupling unit configured for coupling the outlet end of the cannula. The transfer tube is flexible and hollow and comprises an outlet end and an inlet end configured for being fluidly coupled to the cannula outlet end.

The kit-of-parts can also comprise a separation unit, comprising a separator vessel, a filter carousel, a filter inlet opening, a filtrate outlet opening, and a retentate outlet opening. The separator vessel comprises a separator wall defining a geometry of revolution about a separator axis (Z22) defining a separator inner volume closed by a separator lid. The filter carousel is mounted in the separator inner volume and comprises a filter floor and a filter wall having a geometry of revolution about the separator axis (Z22). The filter floor and/or the filter wall comprises one or more filter elements dividing the separator inner volume into a retentate volume and a filtrate volume. The filter inlet opening is provided in the separator lid and opens in the retentate volume. The filtrate outlet opening is in fluid communication with the filtrate volume and is configured for evacuating liquids and fines which passed through the filter elements. The retentate outlet opening is in fluid communication with the retentate volume and is configured for evacuating the coarse fraction retained by the filter elements.

The filter carousel can be rotatably mounted in the separator inner volume, comprising a driving coupling element configured for driving a rotation of the filter carousel about the separator axis (X22) relative to the separator vessel when coupled to a motor. The rotation of the filter carousel generates centrifugal forces driving the flow of the liquids and fines present in the liposuction aspirate through the filter elements. Alternatively, the filter carousel can be static relative to the separator vessel and the flow of the liquids and fines present in the liposuction aspirate through the filter elements can be driven by a source of vacuum. The source of vacuum can also be used with a rotating filter carousel, but in this case, vacuum is not essential.

The separation unit preferably comprises a scraping unit comprising scraping blades configured for scraping the filter wall and/or the filter floor upon rotation of the scraping unit relative to the separator wall (22w). The relative rotation of the scraping blades relative to the filter wall prevents accumulation of coarse material against the filtering elements, causing a pressure drop across the filter elements and blocking access to the filtering elements to the liquids and fines present in the liposuction aspirate. The relative rotation can be obtained by rotating the scraping blades relative to a static filter carousel. Blocking a rotation of the scraping blades with a rotating filter carousel, or rotating the scraping blades at a rotation speed different from the rotation speed of the filter carousel. The scraping unit can be coupled to a clutch release mechanism configured for moving from a coupled configuration to an decoupled configuration, wherein,
in the coupled configuration, the scraping unit (26) is coupled to the filter carousel such that the scraping unit does not rotate relative to the filter carousel, and
in the decoupled configuration, the scraping unit is decoupled from the filter carousel and can rotate about the filter axis (X22) relative to the filter carousel, so that the scraping unit can scrape the surfaces of the one or more filter elements as the scraping unit rotates relative to the filter carousel.

In a preferred embodiment, the size-classifier is stacked on top of the separation unit with the classifier floor replacing the separator lid, thus forming a compact assembly having a reduced footprint in an operating theatre. The driving coupling element of the classifier unit also drives the rotation of the filter carousel. A clutch release mechanism can be provided, allowing the driving coupling element to drive independently or in combination two by two the carousel, the filter carousel and the scraping unit.

The kit-of-parts can also comprise a fluidization unit for fluidising an adipose tissue and comprising a first chamber and a second chamber separated from the first chamber by a filtering member, first and second piston pumps for driving back and forth a flow of liposuction aspirate between the first and second chambers. The first piston pump is fluidly coupled to the first chamber via a first aperture. Similarly, the second piston pump is fluidly coupled to the second chamber via a second aperture. The first and second piston pumps are configured for being activated in opposition of phase, allowing forcing adipose tissue to flow back and forth from one of the first and second chamber to the other one of the first and second chambers through the filtering unit to fluidify the adipose tissue.

The filtering member preferably comprises at least two filters. A first filter has openings larger than a second filter and so on. In this embodiment, the at least two filters are mounted on separate windows of a support frame (32s) configured for allowing each of the at least two filters to be brought successively into a position separating the first chamber from the second chamber. For example, the support frame can be moved to bring each window loaded with one of the at least two filters in the position separating the first chamber (31c) from the second chamber by rotation or by translation of the support frame.

The present invention also concerns a liposuction assembly obtained by assembling the components of the kit-of parts defined supra coupled to one another by transfer tubes.

In a first embodiment, beside the liposuction device, the liposuction assembly comprises a filtering unit and a size-classifier, wherein
a transfer tube is coupled in fluid communication to the cannula outlet of the liposuction unit and the filter inlet opening,
a transfer tube is coupled in fluid communication to the retentate outlet opening of the retentate unit and the classifier inlet opening
a rotation transmission cable is coupled to the liposuction unit and, optionally to the carousel of the size-classifier and/or to the filter carousel of the filtering unit.

Note that the carousel of the size-classifier must rotate, which can be driven by the motor via the rotation transmission cable, or by means of magnets applied to the classifier wall forming a stator and to the filter carousel wall, forming the rotor of an electric motor. The filter carousel may be rotated or be static relative to the filtering vessel. If it rotates, the same mechanisms as described supra for the carousel of the size-classifier can be applied to the filter carousel.

In an alternative embodiment, the liposuction assembly comprises also a fluidization unit as discussed supra, located between the filtration unit and the size-classifier, wherein
a transfer tube is coupled in fluid communication to the cannula outlet of the liposuction unit and the filter inlet opening,
a transfer tube is coupled in fluid communication to the retentate outlet opening of the retentate unit and the first piston pump,
a transfer tube is coupled in fluid communication to the second piston pump to the classifier inlet opening of the size-classifier,
a rotation transmission cable is coupled to the liposuction unit and, optionally to the carousel of the size-classifier and/or to the filter carousel of the filtering unit,

The liposuction assembly comprises first and second fluidisation valves located upstream and downstream of the fluidization unit (32) to allow the latter to be isolated from the rest of the assembly and to allow the flowing back-and-forth of adipose tissue enclosed between the first and second valves.

### BRIEF DESCRIPTION OF THE FIGURES

For a fuller understanding of the nature of the present invention, reference is made to the following detailed description taken in conjunction with the accompanying drawings in which:

FIG. 1(a): shows an embodiment of liposuction assembly comprising a size-classifier according to the present invention.

FIG. 1(b): shows an alternative embodiment of liposuction assembly comprising a size-classifier according to the present invention.

FIGS. 2(a) and 2(b): show a top view and a perspective view of an embodiment of size-classifier according to the present invention.

FIGS. 3(a) and 3(b): show a top view and a perspective view of an alternative embodiment of size-classifier according to the present invention.

FIGS. 4(a) and 4(b): show a top view and a perspective view of yet an alternative embodiment of size-classifier according to the present invention.

FIGS. 5(a) and 5(b): show an exploded view and a side cut view of a size-classifier according to the present invention.

FIG. 6(a): shows an exploded view of a separation unit according to the present invention.

FIGS. 6(b) and 6(c): show two side cut views of the separation unit of FIG. 6(a), with the engagement tabs of the scraping blade engaged and disengaged to drive or not the rotation of the filtering unit.

FIGS. 7(a) and 7(b) show an exploded view and a side cut view of an assembly of a size-classifier stacked on top of a separation unit according to the present invention.

FIGS. 8(a) and 8(b) show an exploded view and a side cut view of a fluidising unit suitable for an assembly according to the present invention.

FIGS. 8(c) and 8(d) show two embodiments of a fluidising unit suitable for an assembly according to the present invention.

### DETAILED DESCRIPTION OF THE INVENTION

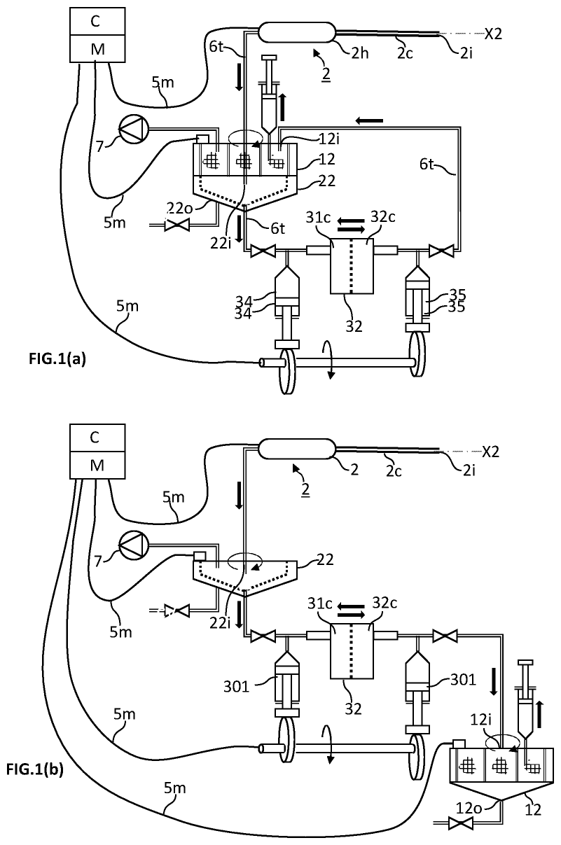

As illustrated in FIGS. 1(a) and 1(b), the present invention concerns a kit of parts and an assembly forming an apparatus for liposuction of adipose tissue and size separation of a liposuction aspirate, comprising, (A) a liposuction device (2), (B) a transfer tube (6t) which is flexible and hollow, comprising an outlet end and an inlet end configured for being fluidically coupled to the liposuction device (2), (C) a source of vacuum (7) in fluid communication with the extraction tube for driving an extraction of the adipose tissue (2), and (D) a size-classifier (12) according to the present invention, discussed in more detail below and configured for separating the liposuction aspirate into fractions of specific size ranges.

The liposuction device (2) comprises a cannula (2c) which is substantially linear, hollow, and elongated, comprising an inner lumen extending along a cannula axis (X2) from an inlet end (2i), provided with one or several openings for drawing adipose tissue into said lumen, to an outlet end located at an opposite end of the cannula. The transfer tube (6t) is coupled to and in fluid communication with the outlet end of the cannula. The liposuction device also comprises a handpiece (2h) comprising a cannula coupling unit configured for coupling the outlet end of the cannula.

The gist of the present invention is the size-classifier (12) configured for dividing adipocyte cells into specific size fractions, and comprising a classifier vessel (12v), a carousel (14), a classifier inlet opening (12i) and a classifier outlet opening (120), and a driving coupling element (16c) configured for driving a rotation of the carousel (14) relative to the classifier vessel (12v) when coupled to a motor (M).

The classifier vessel (12v) has a classifier floor (120, a classifier wall (12w) having a geometry of revolution about a classifier axis (Z12) and an open end closed by a classifier lid (13) and defining a classifier inner volume.

The carousel (14) is rotatably mounted in the classifier inner volume to rotate about the classifier axis (Z12), and comprises,
a carousel vessel (14v) having a carousel floor (14f, a carousel wall (14w) impervious to liquids, which has a geometry of revolution about the classifier axis (Z12) and an open end sealed by a membrane (14m), defining a carousel inner volume, wherein
the carousel innervolume is divided into a plurality of compartments (14.1-14.n) separated from one another by compartment walls, each compartment wall comprising a transverse wall portion which is transverse to a radial direction of the carousel wall (14w),

The carousel wall (14w) is preferably cylindrical (i.e., formed by rotating a straight line about the classifier axis (Z12), or can have any other geometry of revolution formed by rotating a curved line or a segmented line. As long as the carousel (14) can freely rotate inside the classifier vessel (12v), the geometry of revolution is acceptable. It is preferred for reasons of manufacturing to have a cylindrical carousel wall (14w) coaxial with and inscribed in a cylindrical classifier wall (12w).

The classifier inlet opening (12i) is provided on the classifier lid (13) in registration with the carousel and configured for giving a needle direct access to the membrane (14m), and the classifier outlet opening (120) is located in the classifier floor (12f and is configured for evacuating liquids.

The transverse wall portion separating a first compartment (14.1) from a second compartment (14.2) comprises a mesh of first opening (R1). The transverse wall portion separating an i^{th} compartment (14.i) from a (i+1)^{th} Compartment (14.(i+1)), with i=2 to (n−1), comprises a mesh of i^{th} opening (Ri), smaller than an (i−1)^{th} opening (R(i−1)) of a transverse wall portion separating an (i−1)^{th} compartment (14.(i−1)) from the i^{th} compartment (14.i), (i.e., R(n−1)<R(n−2) . . . <Ri< . . . <R1).

In use, adipose cells are deposited into the first compartment (14.1) through the classifier inlet (12i). The carousel (14) is rotated relative to the classifier vessel (12v) about the classifier axis (Z12). The centrifugal force thus created presses the adipose cells against the mesh of first aperture (R1) of the transverse wall portion separating the first compartment (14.1) from the second compartment (14.2). The particles of size larger than the first aperture (R1) are retained in the first compartment (14.1) as retentate, whilst the smaller particles and liquids traverse the first mesh and penetrate into the second compartment (14.2) as filtrate. This is repeated in each successive compartment (14.i), wherein the particles larger than the i^{th} mesh of i^{th} aperture (Ri) are retained in the i^{th} chamber (14.i) as retentate and the smaller particles and liquids traverse the i^{th} mesh into the (i+1)^{th} compartment as filtrate, driven by the centrifugal force. This is repeated until reaching the n^{th} compartment (14.n).

The carousel floor of the n^{th} compartment (14f.n) comprises an opening in fluid communication with the classifier floor (120 and with the outlet opening (120). The filtrate formed by the liquids and particles sufficiently small to traverse the (n−1)^{th} mesh to penetrate into the n^{th} compartment are evacuated through the classifier outlet opening (120). The n^{th} transverse wall separating the n^{th} compartment (14.n) from the first compartment (14.1) is impervious to liquids, forcing all cells and liquids which reached the n^{th} compartment (14.n) to be evacuated through the classifier outlet opening (120).

### The Size Classifier (12)

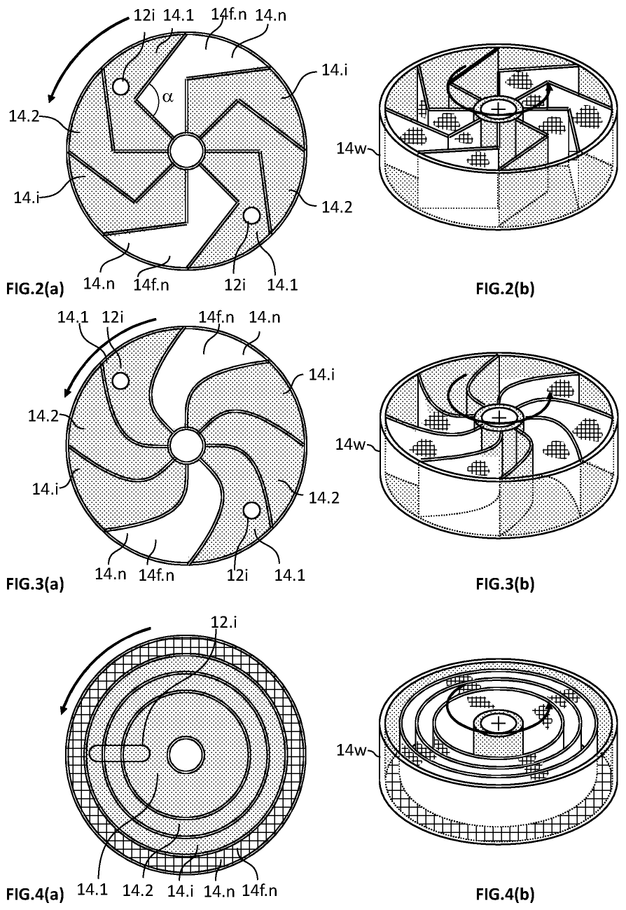

As discussed supra and illustrated in FIGS. 2(a) and 2(b) to 5(a) the classifier vessel (12v), with its classifier floor (12f and classifier wall (12w) and topped by the classifier lid (13) define the inner volume of the classifier vessel (12v), as well as the outer boundaries of the size-classifier (12) of the present invention. The classifier wall (12w) is preferably cylindrical with a substantially circular floor (120 whose central axis of revolution is formed by the classifier axis (Z12). The classifier floor (120 comprises the classifier outlet opening for evacuating out of the size classifier all liquids and very small particles that reached the n^{th} compartment (14.n).

Inside the classifier vessel (12v) the carousel (14) is mounted such as to freely rotate within the inner volume about the classifier axis (Z12). The carousel (14) is formed by a carousel vessel (14v) defining an inner volume, separated into a plurality of compartments separated by compartment walls. The inner volume of the carousel vessel (14v) is defined by a substantially carousel wall (14w) of axis of revolution formed by the classifier axis (Z12), a carousel floor (140, and preferably a membrane (14m) sealing a free end of the cylindrical wall (14v) opposite the carousel floor (14f. The carousel wall (14w) is impervious to the liquids contained in the liposuction aspirates.

The compartments (14.i, i=1−n) are separated from one another by compartment walls comprising at least a section forming a transverse wall portion which is transverse, preferably perpendicular to a radial direction of the carousel (14). The transverse wall portion separating an i^{th} compartment (14.i) from a (i+1)^{th} compartment (14.(i+1)), with i=1 to (n−1), comprises a mesh of i^{th} opening (Ri), smaller than an (i−1)^{th} opening (R(i−1)) of a transverse wall portion separating a (i−1)^{th} compartment (14.(i−1)) from the i^{th} Compartment (14.i), with the first compartment (14.1) having the largest opening (R1), (i.e., R(n−1)<R(n−2) . . . <Ri< . . . <R1).

As illustrated in FIGS. 2(a) & 2(b) and 3(a) & 3(b), the compartment walls can extend substantially radially from a central hub and change orientation to reach the carousel wall (14w) transverse to the radial direction of the carousel (14), forming with the radial direction an angle (α) preferably comprised between 45 and 120°, more preferably between 6° and 90°. The change of direction can form a corner, defining a segmented compartment wall as shown in FIGS. 2(a) & 2(b). Alternatively, the change of direction can be smooth defining a curved compartment wall as illustrated in FIGS. 3(a) & 3(b).

In an alternative embodiment illustrated in FIGS. 4(a) & 4(b), the compartment walls are cylindrical walls coaxial with the classifier axis (Z12) and of different radii, defining annular compartments. This embodiment has the advantage that the adipose cells can be introduced trough the classifier inlet (12i) into the first compartment (14.1) for any angular position of the carousel relative to the position of the classifier inlet (12i). This means that new material can be fed to the first compartment (14.1) as the carousel is rotating, until one or more compartments (14i) are filled with adipose cells of the desired size range and must be emptied.

In the embodiments illustrated in FIGS. 2(a) & 2(b) and 3(a) & 3(b) comprising compartment walls (14i) extending from a central hub of the carousel (14) to the carousel wall (14w), the transverse wall portion separates the n^{th} compartment (14.n) from the first compartment (14.1). The transverse wall portion of the n^{th} Compartment wall is impervious to liquids to prevent passage of any liquids or solids from the n^{th} compartment (14.n) to the first compartment. The carousel floor of the n^{th} compartment (14f.n) comprises an opening to allow any material that reached the n^{th} compartment to fall onto the classifier floor (120 and to be evacuated through the outlet opening (12o). The opening in the carousel floor of the n^{th} compartment (14f.n) can be formed by an absence of any floor, or by a grid or mesh of openings larger than or equal to the (n−1)^{th} openings (R(n−1)) of the transverse wall portion of the (n−1)^{th} compartment wall, or by an aperture in the floor of size sufficiently large to evacuate the material reaching the n^{th} compartment (14.i).

In the embodiments illustrated in FIGS. 2(a) & 2(b) and 3(a) & 3(b), the carousel (14) can comprise several series of n compartments (14.1 to 14.n) distributed around the classifier axis (Z12). For example, in FIGS. 2(a) and 3(a), carousels comprising two series of n compartments are illustrated. Depending on the volume of the compartments relative to the size of the carousel, the inner volume of the carousel (14) can be divided into one, two, three, four, or more series of n compartments distributed around the classifier axis (Z12). If the carousel comprises more than one series of n compartments, care must be taken to feed the first compartment (14.1) of each series with adipose cells prior to starting size separation.

In the embodiment illustrated in FIGS. 4(a) and 4(b), wherein the compartments (14i) are separated from one another by concentric compartment walls of increasing radius, the n^{th} compartment wall is formed by the carousel wall (14w), which may or may not be impervious to liquids. If, on the one hand, the carousel wall (14w) is impervious to liquids, the carousel floor of the n^{th} compartment (14f.n) comprises an opening or is formed by a grid or mesh of opening larger than the n^{th} opening (Rn) to allow any material that reached the n^{th} compartment to fall onto the classifier floor (120 and be evacuated through the outlet opening (120). If, on the other hand, the carousel wall (14w) is formed by a mesh, the liquid is evacuated through the carousel wall (14w) by the centrifugal force to fall into the carousel vessel (12v) and out thereof through the classifier outlet opening (120). In this latter case, the carousel floor of the n^{th} compartment (14f.n) may or may not comprise an opening or be formed by a grid or mesh.

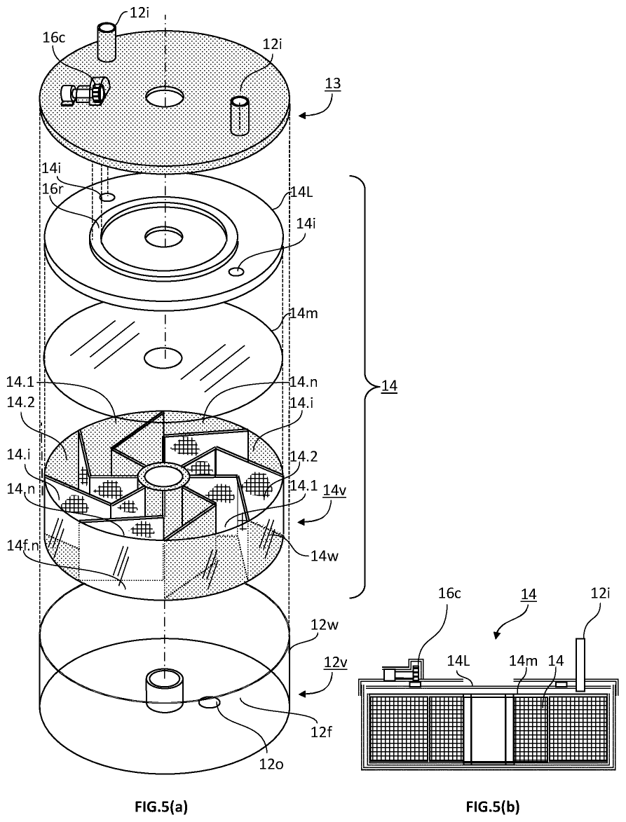

The driving coupling element (16c) is configured for driving a rotation of the carousel (14) relative to the classifier vessel (12v) when coupled to a motor (M). Any driving coupling element (16c) able to drive the rotation of the carousel (14) can be used in the size-classifier of the present invention. For example, as shown in FIGS. 5(a) and 5(b), the driving coupling element (16c) can be formed by a drive gear bearing whose position is fixed relative to the classifier vessel (12v) which is engaged on a rack (16r) whose position is fixed relative to the carousel (14). The drive gear bearings and rack form a rack and pinion system configured for rotating the carousel (14) relative to the classifier vessel (12v). As illustrated in FIGS. 5(a) and 5(b), the rack can form a circle centred on the classifier axis (Z12) provided on a carousel lid (14L) fixed to the free end of the carousel wall (14w). The drive gear bearings (16c) can be fixed to the classifier lid (12L). Alternatively, the rack (16r) can surround an outer perimeter of the carousel wall and the drive gear bearings (16c) can be fixed the classifier wall (12w). In an alternative embodiment, magnets, such as induction magnets, permanent magnets or combinations of both, can be provided on the classifier wall (12w) to form the stator of an electric motor and on the carousel wall (14w) to form the rotor of the electric motor. This embodiment has the advantage of not requiring a rotation transmission cable (5m) coupled to an external motor (M), since the size classifier can form the motor itself. This embodiment also facilitates maintenance of the sterile environment in the inner volume of the size classifier (12), as no contact is required between the magnets of the rotor and stator to drive the rotation of the carousel (14). The carousel (14) can be rotated at a rotational rate comprised between 100 and 5000 rpm, preferably between 500 and 3000 rpm, more preferably between 700 and 2000 rpm.

In use, the first compartment (14.1) must face the classifier inlet opening (12i). Depending on the design of the classifier carousel, this is always the case for annular compartments as illustrated in FIGS. 4(a) & 4(b)I, or the classifier carousel must be rotated in case of partly radial compartment walls as shown in FIGS. 2(a) & 2(b) and 3(a) & 3(b). Adipose cells are deposited into the first compartment (14.1) through the classifier inlet (12i). If a membrane (14m) seals the inner volume of the carousel (14), a needle can be used to puncture it. The carousel (14) is rotated relative to the classifier vessel (12v) about the classifier axis (Z12). The centrifugal force thus created drives the adipose cells against the mesh of first aperture (R1) of the transverse wall portion separating the first compartment (14.1) from the second compartment (14.2). The particles of size larger than the first aperture (R1) are retained in the first compartment (14.1) as retentate, whilst the smaller particles and liquids traverse the first mesh and enter into the second compartment (14.2) as filtrate. This is repeated in each successive compartment (14.i), wherein the particles larger than the i^{th} mesh of i^{th} aperture (Ri) are retained in the i^{th} chamber (14.i) as retentate and the smaller particles and liquids traverse the i^{th} mesh into the (i+1)^{th} compartment as filtrate, driven by the centrifugal force. As the carousel (14) rotates, this mechanism is repeated sequentially until reaching the n^{th} compartment (14.n). When each compartment contains the adipose cells of corresponding size ranges, a volume of adipose tissue of a given size range can be collected with a syringe through the classifier inlet opening (12i) (which then functions as an outlet). Again, if the compartments (14.i) extend from a central hub to the classifier wall as shown in FIGS. 2(a) and 3(a), the carousel must be rotated until the corresponding compartment faces the classifier outlet opening (12i). If, on the other hand, the compartments are concentric annular compartments, a single classifier inlet opening (12i) which is elongated and spanning in a radial direction over the first to (n−1)^{th} compartment or a series of (n−1) openings distributed radially over each annular compartment suffices to have access to the adipose tissues of all size fractions (cf. FIG. 4(a)).

The carousel floor of the n^{th} compartment (14f.n) comprises an opening in fluid communication with the classifier floor (120 and with the outlet opening (120). The filtrate formed by the liquids and particles sufficiently small to traverse the (n−1)^{th} mesh to penetrate into the n^{th} compartment are evacuated through the classifier outlet opening (120). If the compartment walls extend from a central hub of the carousel (14) to the carousel wall (14w), the n^{th} transverse wall separating the n^{th} compartment (14.n) from the first compartment (14.1) is impervious to liquids, forcing all cells and liquids which reached the n^{th} compartment (14.n) to be evacuated through the classifier outlet opening (120).

The first opening R1 can have a first diameter comprised between 200 and 1000 μm, preferably between 400 and 800 μm, and the (n−1)^{th} opening (R(n−1)) can have a (n−1)^{th} diameter comprised between 10 and 200 μm, preferably between 20 and 100 μm, more preferably between 30 and 70 μm. The (n−1)^{th} transverse wall portion has the smallest opening (R(n−1)) The number n of compartments in a series can be comprised between 3 and 6 (i.e., n=3-6), and is preferably equal to 4 or 5 (i.e., n=4 or 5).

As discussed supra, the compartment walls can have any geometry, provided they comprise the transverse wall portion transverse to the radial direction from the classifier axis (Z12), and that they divide the inner volume of the carousel (14) into compartments (14i) arranged sequentially relative to the direction of rotation of the carousel (14), such that it is not possible to pass from the (i−1)^{th} compartment (14.(i−1)) to the (i+1)^{th} compartment (14.(i+1)) without first passing through the i^{th} compartment (14.i).

The size-classifier (12) of the present invention is advantageously used in a liposuction assembly, comprising a liposuction device (2), a size-classifier (12) according to the present invention, and one or more transfer tubes (6t) in (indirect) fluid communication between the liposuction device and the size-classifier.

### Liposuction Kit-of-Parts and Liposuction Assembly

The present invention also concerns a kit-of-parts and an assembly for extracting adipose tissues from a body, treating the adipose tissue to separate adipose cells into specific size fractions for further topical uses. As illustrated in FIGS. 1(a) and 1(b), the kit-of-parts and assembly comprises a liposuction device (2), a size-classifier (12) as defined supra, and one or more transfer tubes (6t) in (indirect) fluid communication between the liposuction device and the size-classifier. It preferably comprises a separation unit (22) and a fluidisation unit (32), also in fluid communication with one another by means of transfer tubes (6t) as shown in FIGS. 1(a) & 1(b).

### Liposuction Device (2)

The liposuction device (2) comprises a cannula (2c) and a handpiece (2h) and is connected to a vacuum pump (7). As illustrated in FIGS. 1(a) & 1(b), the liposuction device comprises a substantially linear, hollow, elongated cannula (2c) with an inner lumen extending along a cannula axis, X2, from a first inlet end (2i), provided with one or several openings for drawing adipose tissue into said lumen, to a second, outlet end, located at the opposite end of the elongated body. The cannula is coupled to the handpiece (2h) by means of fixing means to the handpiece (2h).

The present invention is not restricted to any choice of a particular liposuction device. It is preferred, however, that the liposuction device preserves the integrity of the adipose cells extracted therewith. Powered handpieces (2h) are preferred, which can impart to the inlet end (2i) of the cannula a vibrational movement comprising at least a linear component of a back-and-forth reciprocal movement along the cannula axis, X2, at a frequency of for example, 1 to 500 Hz, preferably between 20 and 70 Hz, more preferably between 50 and 55 Hz. To preserve the integrity of the adipose cells thus aspirated, it is preferred that the vibrational movement also comprises an orbital component about the cannula axis, X2. The orbital component of the vibrational movement can be substantially elliptical. The combination of the longitudinal and orbital components of the vibrational movement is often referred to as nutation. It was observed that, when using a manual or other (non-nutational) powered liposuction device, the adipose tissues were collected in the form of lumps formed by a rather large number of adipose cells. With a nutational powered handpiece, it was concluded that the adipose tissues were extracted in the form of a mixture of substantially smaller lumps and of individual cells, which is advantageous for the survival of the extracted adipose cells. Examples of liposuction devices (2) imparting a nutational movement to the tip of the cannula are described in WO9844966, U.S. Pat. No. 6,336,925, or WO2021144602. Their use is herein preferred in the assembly of the present invention.

In case of a powered handpiece (2h), it can be powered pneumatically as described e.g., in WO9844966. Alternatively and preferably, as illustrated in FIGS. 1(a) & 1(b), an external motor (M) can be used instead, coupled to the liposuction device (2) by a rotation transmission cable (5m) configured for transmitting a rotational torque to the liposuction device. In this embodiment, the liposuction device must be equipped with a mechanism configured for transforming the rotational torque transmitted by the rotation transmission cable (5m) into a reciprocal, preferably a nutational vibration to the tip of the cannula (2c). An example of such mechanism is described in WO2021144602.

Before transferring the adipose tissues extracted by the liposuction device (2) to the size-classifier (12), it is preferred to pre-treat and wash the liposuction aspirate to eliminate all undesired tissues, such as fibrous connective tissues, and liquids, such as oils and blood and to break agglomerates into individual adipose cells or into agglomerates of substantially smaller dimensions. This can be achieved by including treatment units between the liposuction device (2) and the size-classifier (12) fluidly coupled to one another by transfer tubes (6t). The transfer tubes (6t) are flexible and hollow and comprise an inlet end and an outlet end configured to be coupled in fluid communication to two components of the assembly. The treatment units can include for example, a separation unit (22) and a fluidization unit (32) as illustrated in FIGS. 1(a) & 1(b), 6(a) to 6(c) and 7(a) & 7(b).

### Separation Unit (22)

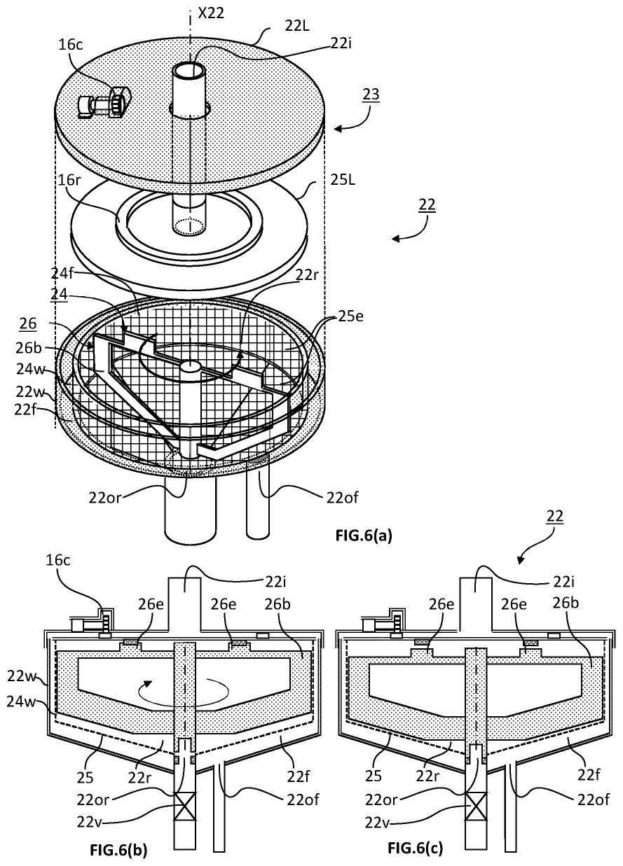

A separation unit (22) can be provided directly downstream of the liposuction device (2) to eliminate most undesirable tissues and the liquids. As illustrated in FIGS. 6(a) to 6(c), the separation unit (22) can comprise a separator vessel (22v), a filter carousel (24) comprising a mesh or a grid of predefined opening size. To allow the liposuction aspirate to flow through the separation unit (22), it is provided with,
a filter inlet opening (22i) for admitting the liposuction aspirate into the separation unit (22),
a filtrate outlet opening (22of) to dispose of the liquids and other undesired tissues which passed through the filter carousel (24), and
a retentate outlet opening (22or) for flowing the adipose cells retained in the filter carousel (24) to a next treatment unit of the assembly.

Separation of tissues in the separation unit can be driven by a vacuum pump (7), preferably the same used to drive the aspiration through the cannula (2c) of the liposuction device (2). In this embodiment, the carousel may, but does not need to rotate relative to the separator vessel (22v). A vacuum opening for coupling the vacuum pump (7) to the inner volume of the separator vessel (22v) should be provided, preferably on the classifier lid (13). Alternatively, separation can be driven by centrifugal forces created by the rotation of the filter carousel (24) relative to the separator vessel (22v). To drive the rotation of the filter carousel, a driving coupling element (16c) coupled to a motor by means of a rotation transmission cable (5m) or formed by magnets (not shown) can be attached to the separation unit (22). The magnets can be induction magnets or permanent magnets.

The separator vessel (22v) may be a beaker provided with a separator lid (23) sealed thereon by welding or gluing. The separator vessel (22v) has a separator floor (220, a separator wall (22w) and an open end closed by the separator lid (23) thus defining a separator inner volume. If separation is driven by vacuum, the geometry of the separator wall (22w) is not restricted. On the other hand, in case separation is driven by centrifugal forces created by the rotation of the filter carousel (24), the separator wall (22w) should preferably have a geometry of revolution about a separator axis (Z22), preferably a cylindrical geometry. The filter inlet opening (22i) is preferably provided on the separator lid (23).

As shown in FIG. 6(a), the filter carousel (24) is accommodated in the inner volume of the separator vessel (22v). The filter carousel (24) can comprise a filtering frame (240 supporting one or more filter elements (25e) dividing the filter inner volume into a retentate volume (22r) upstream of the filter elements (25e), and a filtrate volume (220 downstream of the filter elements (25e). The filter elements (25e) have a given opening to retain in the retentate volume solids larger than the given opening The filter carousel can comprise a filter floor and preferably filter walls (24w) surrounding the filter floor, thus forming a basket defining the retentate volume with filter elements (25e) tiling or forming at least a portion of the filter floor and/or of the filter wall. In case separation is driven by vacuum, the filter elements (25e) are preferably concentrated on the filter floor. The vacuum pump (7) can be coupled to the inner volume of the separation unit with an opening in the separator lid (23), as shown in FIGS. 1(a) and 1(b).

In a preferred embodiment, separation is driven by centrifugal forces created by the rotation of the filter carousel (24) about the separator axis (X22). The rotation can be driven by a driving coupling element (16c) configured for driving a rotation of the filter carousel (24) about the separator axis (X22) relative to the separator vessel (22v) when coupled to a motor (M) or forming an electric motor with stator and rotor. In this embodiment, the presence of filter elements (25e) on the filter wall (24w) is essential as the liposuction aspirate is pushed against the filter wall by centrifugal forces, wherein the solids of size larger than the opening of the filter elements remain in the retentate volume, whilst liquids and small tissues pass through the filter elements (25e) into the filtrate volume. In this embodiment, the filter wall is preferably cylindrical (i.e., formed by rotating a straight line about the separator axis (Z22)), or can have any other geometry of revolution formed by rotating a curved line or a segmented line. As long as the filter carousel (24) can freely rotate inside the separator vessel (22v), the geometry of revolution is acceptable. It is preferred for reasons of manufacturing to have a cylindrical filter wall (24w) coaxial with and inscribed in a cylindrical separator wall (22w). As discussed in reference to the rotation of the carousel (14) of the size-classifier supra, magnets can be provided on the separator wall (22w) to form the stator of an electric motor and on the filter carousel wall (24w) to form the rotor of the electric motor. This embodiment has the advantage of not requiring a rotation transmission cable (5m) coupled to an external motor (M), since the separation unit (22) can form the motor itself.

The filter floor preferably includes the retentate outlet opening (22or), preferably located at a bottom of a slope. For example, the floor can be conical forming a funnel, as shown in FIGS. 6(b) and 6(c). The retentate opening communicates with a tube provided with a separator valve (22v), which can be moved from,
a closed position to retain the retentate in the retentate volume during the separation process allows evacuation of the retentate towards a next treatment unit of the assembly to
an open position to allow passage of the adipose cells retained by the filter elements (25e) when all liquids and small debris are considered to have gone through the filter elements (25e) into the filtrate volume. The retentate can thus be flowed to a next treatment unit.

The liquids and small debris forming the filtrate can be evacuated out of the separation unit (22) through the filtrate opening (22of) provided on the separator floor (220 as clearly visible in FIGS. 6(b) & 6(c).

In a preferred embodiment, the separation unit (22) comprises a scraping unit (26) comprising scraping blades (26b) configured for scraping the filter wall (24w) and filter floor upon rotation of the scraping unit (26) relative to the filter carousel (24). The scraping unit (26) is configured for rotating about the separator axis (X22) relative to the filter carousel (24). If the filter carousel is static relative to the separator vessel (22v) during separation, scraping of the filter carousel is obtained by rotating the scraping unit (26) about the separator axis (X22). If, on the other hand, the filter carousel is configured for rotating about the separator axis (X22) relative to the separator vessel (22v), scraping of the filter carousel is obtained by blocking the scraping unit (26) or by varying the rotation speed of the scraping unit (26) about the separator axis (X22) whilst the filter carousel (24) keeps rotating.

To allow the scraping unit (26) to rotate or be static relative to the filter carousel (24) when scraping is required or not, the scraping unit (26) can be coupled to a clutch release mechanism configured for moving from a coupled configuration to a decoupled configuration, wherein,
in the coupled configuration, the scraping unit (26) is coupled to the filter carousel (24) such that the scraping unit (23) does not rotate relative to the filter carousel (24), and
in the decoupled configuration, the scraping unit (26) is decoupled from the filter carousel (24) and can rotate about the filter axis (X22) relative to the filter carousel (24), so that the scraping unit (26) can scrape the surfaces of the one or more filter elements (25e) as the scraping unit (26) rotates relative to the filter carousel (24).

As shown in FIGS. 6(a) to 6(c), the clutch release mechanism can comprise engagement tabs (26e) which can engage or not mating elements of the filter carousel (24), by moving the scraping unit (26) along the separator axis (X22).

The assembly of the present invention can comprise additional treatment units including, for example, a fluidisation unit (32) as illustrated in FIGS. 1(a) & 1(b) and 8(a) & 8(b).

### Fluidisation Unit (32)

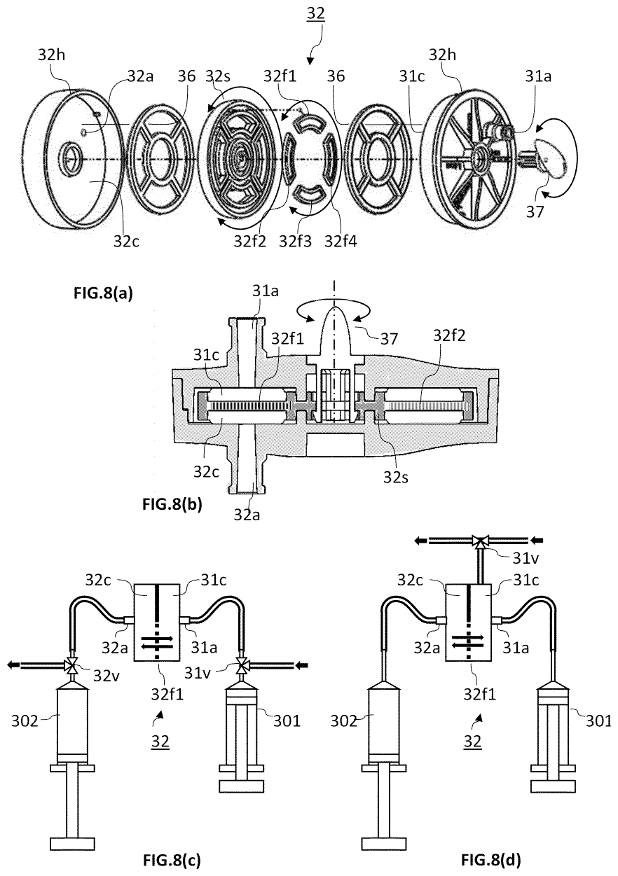

A fluidisation unit (32) is configured for disaggregating lumps of adipose cell aggregates, loosely held together by secondary forces (hydrogen bonds, London or Keaton forces, and the like) and thus yielding a more homogeneous aspirate formed of substantially smaller aggregates and of individual adipose cells, with enhanced flowability. As illustrated in FIGS. 8(a) to 8(d), the fluidisation unit (32) comprises first and second chambers (31c, 32c) separated from one another by a filtering member (32fi). First and second piston pumps (301, 302) are configured for driving a back-and-forth passage of the liposuction aspirate through the filtering member (32fi) between the first and second chambers (31c, 32c).

The first chamber (31c) comprises a first aperture (31a) fluidly coupled to the first piston pump (301), and the second chamber (32c) comprises a second aperture (32a) fluidly coupled to the second piston pump (302). During a fluidisation operation, the first and second piston pumps (301, 302) are configured for being activated in opposition of phase, allowing forcing adipose tissue to flow back and forth from one of the first and second chambers to the other one of the first and second chambers through the filtering member (32fi) to fluidify the adipose tissue by disaggregating the liposuction aspirate aggregates into a mixture of individual adipose cells and aggregates of adipose cells of smaller dimensions. It is known to a person skilled in the art how to control the actuation in phase opposition of the first and second piston pumps (301, 302). FIGS. 1(a) and 1(b) illustrate an example using rotating cams coupled to the pistons of the first and second piston pumps (301, 302). The rotation of the rotating cams can be driven by an external motor (M) coupled thereto by a rotation transmission cable (5m). The same motor (M) as used for rotating the carousel (14) and filter carousel (24) or scraping unit (26) is used for driving the rotation of the rotating cams. Else a processor can control any activating mechanism to ensure that the first and second piston pumps (301, 302) be operated in phase opposition when required.

As shown in FIG. 8(c), liposuction aspirate can be introduced into and maintained within the fluidisation unit (32) by means of first and second three-way fluidisation valves (31v, 32v) coupled to the first and second piston pumps (301, 302). Alternatively, as illustrated in FIG. 8(d), entry into the fluidisation unit (32) is controlled by a single three-way fluidisation valve (31v) giving access to the first chamber and is maintained in the fluidisation unit by closing the three-way fluidisation valve (31v) and actuating the first and second piston pumps (301, 302) to drive the liposuction aspirate back and forth between the first and second chambers (31c, 32c). It is clear that a three-way valve can be replaced by two or more successive two-way valves to yield the same effect.

In a preferred embodiment, the filtering member (32fi) comprises at least two filters (32f1, 32f2, 32f3, 32f4), a first filter (32f1) having openings larger than a second filter (32f2) and so on, and wherein the filtering member (32fi) is mounted such that each of the at least two filters can be brought successively into a position wherein they separate alone the first chamber (31c) from the second chamber (32c).

As shown in FIGS. 8(a) and 8(b), the at least two filters (32f1, 32f2, 32f3, 32f4) can be mounted on separate windows of a support frame (32s), wherein the support frame can be moved to bring each window loaded with one of the at least two filters (32f1, 32f2, 32f3, 32f4) in the position separating the first chamber (31c) from the second chamber (32c). This can be achieved by rotating the support frame (32s) as shown in FIGS. 8(a) & 8(b). Alternatively, it can be achieved by translation of the support frame (32s), or by a combination of rotation and translation.

Fluidisation of the liposuction aspirate with the present fluidisation unit (32) can proceed as follows. Adipose tissue is flowed into the first chamber (31c) of the fluidisation unit (32). This can be achieved by opening the first fluidisation valve (31v) and drawing vacuum by pulling out the piston of the first piston pump (301). Once the cylinder of the first piston pump (301) is filled with adipose tissue, the first fluidisation valve is closed and the adipose tissue is forced to flow into the first chamber (31c) by pushing the piston of the first piston pump (301) in while pulling the piston of the second piston pump (302) out, and through a first filtering member (32f1) into the second chamber (32c). With each and any of the first and second fluidisation valves (31v, 32v) in a closed position, the adipose tissue can be driven back-and-forth through the first filtering member (32f1) between the first and second chambers (31c, 32c). When it is considered that the adipose tissue has traversed the first filtering member (32f1) a sufficient number of times, a second filtering member (32f2) of openings smaller than the openings of the first filtering member (32f1) replaces the other. In the preferred embodiment, this is made easily by rotating or translating the support frame (32s), as shown in FIG. 8(b). The adipose tissue is flowed back-and-forth through the second filter member (32f2) as described supra with respect to the first filter member (32f1) and a third filter member (32f3) can replace the second filter member (32f2) as described supra, and so on. The interval of time a same filter member is in use before replacing it by a next filter member of smaller openings can be dictated by a predefined number of back-and-forth travelling of the adipose tissue through a given filter member. Alternatively, it can be determined as a function of the evolution of the pressure required to push the piston in to drive the flow through the filter member. A processor can be provided configured for indicating to an operator or controlling that a next filter member must replace a former filter member based on the value of the pressure measured on the first and second piston pumps (301, 302).

When the adipose tissue has passed often enough through the last (finest) filter member (32f4), the corresponding fluidisation valve (31v, 32v) is open to allow the thus fluidised adipose cells to flow out of the fluidisation unit (32). In the embodiment of FIG. 8(c), the first fluidisation valve (31v) remains closed, and the second fluidisation valve (32v) opens. In the embodiment of FIG. 8(d), the first fluidisation valve (31v) is opened.

The first filter member (32f1) is the coarsest filter, with openings of size which can be comprised between 1 mm and 5 mm. The sequence of filter members (32f1-32f4) from the first filter member (32f1) to the n^{th} (=last) filter member (32f4) is characterized by a reduction of the opening size of the successive filter members. The n^{th} (=last) filter member (32f4) is the finest filter member with openings of size which can be comprised between 10 μm and 1 mm.

As shown in FIGS. 1(a) and 1(b), the thus fluidised adipose cells can flow from the fluidisation unit (32) to the size-classifier (12) through a transmission tube (6t). The flow can be driven by one of the first and second piston pumps (301, 302) or by a vacuum pump. It is preferred to not use a vacuum pump downstream of the separation unit (22) to preserve the integrity and moisture of the adipose cells, and to allow addition of a saline solution to the cells if required.

### Stack of the Size-Classifier (12) and Separation Unit (22)

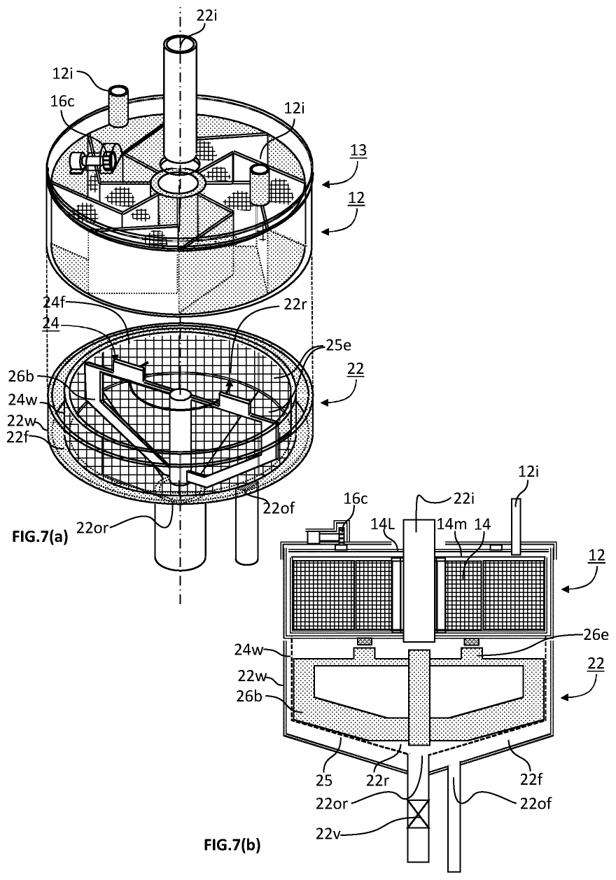

In order to reduce the number of separate treatment units to be assembled and disposed in the operating theatre, some of the treatment units can be combined by stacking them on top of one another. For example, as shown in FIGS. 1(a) and 7(a) & 7(b), the size-classifier (12) and separation unit (22) can be stacked on top of one another to form a compact assembly. These two treatment units are interesting to combine, since both comprise rotating elements, which may require to be coupled to a motor (M) via rotation transmission cables (5m). By combining them, a single rotation transmission cable (5m) can be used to rotate the rotating elements of both size-classifier (12) and separation unit (22). To avoid having to rotate the rotating elements of a treatment unit which is not in use, a clutch release mechanism can be provided to engage or disengage the rotating elements of each of the size-classifier (12) and the separation unit (22). In case rotation of the carousel (14) and the filter carousel (24) is driven by an arrangement of electromagnets and optionally permanent magnets, which can be controlled independently by a processor of a controller (C).

The size-classifier (12) is stacked on top of the separation unit (22) with the classifier floor (120 replacing the separator lid (23). It is preferred to stack the size-classifier (12) on top of the separation unit (22) because free access from above to the individual compartments (14.i) is required after size-separation to retrieve the adipose cells of the desired size ranges. For mechanically controlled rotation, the driving coupling element (16c) of the classifier unit (12) forming e.g., a rack-and-pinion system, also drives the rotation of the filter carousel (24) and/or of the scraping unit (26). A clutch release mechanism can be provided to allow the driving coupling element (16c) to drive independently or in combination two by two the carousel (14), the filter carousel (24) and the scraping unit (26). For electric control of the rotation (with magnets), the rotation of the carousel (14) and of the filter carousel (24) (if it rotates), can be controlled independently. In case of a static filter carousel (24), the scraping unit (26) can be reversibly coupled to the carousel (14) to rotate with it or not.

As shown in FIG. 1(a), liposuction aspirate is flowed from the liposuction device (2) into the separator inlet opening (22i) through a tube isolated from and bypasing the size-classifier (12), directly into the retentate volume of the separation unit (22). The flow is driven by a vacuum pump (7). As for the embodiment wherein the separation unit (22) is separate from the size-classifier, the separation unit is operated independently. Undesired debris and liquids are separated from the adipose cells by forcing the former through the filter carousel (24) driven either by the vacuum pump (7), with a static carousel (24) or by centrifugal forces created by the rotation of the carousel (24) relative to the separator vessel (22v), or by a combination of both. When required, the scraping unit (26) can be rotated relative to the filter carousel (24) (i.e., either the scraping blades (26b) stop rotating with, or rotating at a different rate from the rotating filter carousel (24), or they start rotating relative to a static filter carousel (24)), to prevent accumulation of tissues on the filter elements (25e), which increases the pressure drop across the filter elements (25e).

The liquids and undesired small debris are evacuated through the filtrate outlet opening (22of and the adipose tissue retained in the retentate volume are driven out through the retentate outlet opening (22or) by opening the separator valve (22v) towards a next treatment station, such as the fluidisation unit (32) or straight to the size-classifier (12).

### The Liposuction Assembly in Use

After extraction by the liposuction device (2), the liposuction aspirate is separated in the separation unit (22) as explained supra. The separation unit (22) can be stacked below the size-classifier (12) to form a compact assembly, as illustrated in FIG. 1(a), or it can be separate from the size-classifier (12) as illustrated in FIG. 1(b). The liquids and small debris are removed from the liposuction aspirate in the filter carousel (24), either by vacuum or by centrifugal forces, or both, as discussed supra. The retentate is flowed out of the separation unit (22) for further treatments.

The adipose tissue is preferably flowed into the fluidisation unit (32), driven by the first piston pump (301). Once a sufficient volume of adipose tissue was drawn by the first piston pump (301) into the first chamber (31c) the fluidisation process begins as described supra. At the end of the fluidisation process, the thus fluidised adipose cells can be flowed into the first compartment (14.1) of the size classifier (12). The flow can be driven by one of the first and second piston pumps (301, 302) of the fluidisation unit (32) or by a vacuum pump (7).

It is preferred to not use vacuum downstream of the separation unit (12) for driving the flow of the liposuction aspirate to avoid that the latter dries too quickly. Without vacuum, it is also easier to provide humidification stations, wherein a saline solution can be added to the liposuction aspirate, to clean it and to humidify it, thus enhancing the flowability thereof. The flow of the liposuction aspirate downstream of the separation unit (22) can be driven by the first or second piston pumps (301, 302).

When the adipose cells are in the first compartment, the carousel (14) can be rotated to separate the adipose cells by size ranges by passing them through the meshes of the successive transverse wall portions separating two adjacent compartments (14i). Once the adipose cells are separated into size fractions in the corresponding compartments, the adipose cells or desired size fraction can be collected using a needle for any further use. The adipose cells can be used for liposculpture by injection thereof into a body location of a patient with a lipofilling device (not shown) which is similar to a non-powered liposuction device, with a cannula which is generally thinner than in liposuction devices. Alternatively, the adipose cells of desired size fraction can be retrieved for further treatment, such as isolation and culture of stem cells.

The size-classifier (12) of the present invention is very efficient for reproducibly and easily separating adipose cells into predefined size ranges, particularly suitable for topical applications, including lipofilling in liposculpture applications or further treatment and culture of cells, in particular stem cells. The assembly of the present invention allows specific size fractions of liveable adipose cells to be extracted, cleaned, and isolated in a single apparatus which is entirely sterile and without any contact with human hands.

1. A size-classifier (12) for dividing adipocyte cells into specific size fractions, the size-classifier comprising,
a classifier vessel (12v) having a classifier floor (12f), a classifier wall (12w) having a geometry of revolution about a classifier axis (Z12) and an open end closed by a classifier lid (13) and defining a classifier inner volume,
a carousel (14) rotatably mounted in the classifier inner volume to rotate about the classifier axis (Z12), and comprising,
a carousel vessel (14v) having a carousel floor (14f), a carousel wall (14w) which has a geometry of revolution about the classifier axis (Z12) and an open end defining a carousel inner volume, wherein
the carousel inner volume is divided into a plurality of compartments (14.1-14.n) separated from one another by compartment walls, each compartment wall comprising a transverse wall portion which is transverse to a radial direction of the carousel wall (14w),
a classifier inlet opening (12i) in the classifier lid in registration with the carousel and configured for giving a needle direct access to the compartments (14i),
a classifier outlet opening (12o) in the classifier floor (12f) configured for evacuating liquids,
a driving coupling element (16c) configured for driving a rotation of the carousel (14) relative to the classifier vessel (12v) when coupled to a motor (M),
Characterized in that,
the transverse wall portion separating a first compartment (14.1) from a second compartment (14.2) comprises a mesh of first opening (R1),
the transverse wall portion separating an i^{th} compartment (14.i) from a (i+1)^{th} compartment (14.(i+1)), with i=2 to (n−1), comprises a mesh of i^{th} opening (Ri), smaller than an (i−1)^{th} opening (R(i−1)) of a transverse wall portion separating a (i−1)^{th} compartment (14.(i−1)) from the i^{th} compartment (14.i), (i.e., R(n−1)<R(n−2) . . . <Ri< . . . <R1),
the carousel floor of the n^{th} compartment (14f.n) comprises an opening in fluid communication with the classifier floor (12f) and with the outlet opening (12o).

2. The size-classifier (12) according to claim 1, wherein the first opening (R1) has a first diameter comprised between 200 and 1000 μm and the (n−1)^{th} opening (R(n−1)) has a (n−1)^{th} diameter comprised between 10 and 200 μm.

3. The size-classifier (12) according to claim 1, wherein the compartment walls have one of the following geometries,
the compartment walls extend substantially radially from a central hub and change orientation to reach the carousel wall (14w) transverse to the radial direction of the carousel wall (14w), forming with the radial direction an angle (α); the change of direction can form a corner, defining a segmented compartment wall or can be smooth defining a curved compartment wall, or
the compartment walls are cylindrical walls coaxial with the classifier axis (Z12) and of different radii, defining annular compartments.

4. The size-classifier (12) according to claim 1, wherein the opening in the carousel floor of the n^{th} compartment (14f.n) is formed by,
no floor, or
a grid or mesh of openings larger than or equal to the (n−1)^{th} openings (R(n−1)) of the transverse wall portion of the (n−1)^{th} compartment wall, or
an aperture in the floor.

5. The size-classifier (12) according to claim 1, wherein the driving coupling element (16c) is a pinion of a rack-and-pinion mechanism, wherein the pinion is configured for being coupled to a rotation transmission cable (5m) to a motor (M), or comprises magnets forming an electric motor whose stator is formed by the classifier wall (12w) and the rotor by the carousel wall (14w).

6. A kit of parts for an apparatus for liposuction of adipose tissue comprising,
a liposuction device (2) having:
a cannula (2c) which is substantially linear, hollow, and elongated, comprising an inner lumen extending along a cannula axis (X2) from an inlet end (2i), provided with one or several openings for drawing adipose tissue into said lumen, to an outlet end located at an opposite end of the cannula,
a handpiece (2h) comprising a cannula coupling unit configured for coupling the outlet end of the cannula,
a transfer tube (6t) which is flexible and hollow, comprising an outlet end and an inlet end configured for being fluidly coupled to the cannula outlet end,
a source of vacuum (7) in fluid communication with the extraction tube for driving an extraction of the adipose tissue (2),
characterized in that, the kit-of-parts further comprises a size-classifier (12) according to claim 1.

7. The kit of parts according to claim 6, further comprising a separation unit (22) comprising,
a separator vessel (22v) comprising a separator wall (22w) defining a geometry of revolution about a separator axis (Z22) defining a separator inner volume closed by a separator lid (23), and comprising:
a filter carousel (24) mounted in the separator inner volume and comprising a filter floor and a filter wall (24w) having a geometry of revolution about the separator axis (Z22), the filter floor and/or the filter wall (24w) comprising one or more filter elements (25e) dividing the separator inner volume into a retentate volume (22r), and a filtrate volume (22f),
a filter inlet opening (22i) in the separator lid (23) opening in the retentate volume (22r),
a filtrate outlet opening (22of) in fluid communication with the filtrate volume (22f) and configured for evacuating liquids and fines which passed through the filter elements (25e),
a retentate outlet opening (22or) in fluid communication with the retentate volume (22r) and configured for evacuating the coarse fraction retained by the filter elements (25e).

8. The kit of parts according to claim 7, wherein the filter carousel (24) is rotatably mounted in the separator inner volume, comprising a driving coupling element (16c) configured for driving a rotation of the filter carousel (24) about the separator axis (X22) relative to the separator vessel (22v) when coupled to a motor (M).

9. The kit of parts according to claim 7, wherein the separation unit (22) comprises a scraping unit (26) comprising scraping blades (26b) configured for scraping the filter wall (24w) and/or the filter floor upon rotation of the scraping unit (26) relative to the separator wall (22w), and wherein the scraping unit (26) is coupled to a clutch release mechanism configured for moving from a coupled configuration to an decoupled configuration, wherein,
in the coupled configuration, the scraping unit (26) is coupled to the filter carousel (24) such that the scraping unit (23) does not rotate relative to the filter carousel (24), and in the decoupled configuration, the scraping unit (26) is decoupled from the filter carousel (24) and can rotate about the filter axis (X22) relative to the filter carousel (24), so that the scraping unit (26) can scrape the surfaces of the one or more filter elements (25e) as the scraping unit (26) rotates relative to the filter carousel (24).

10. The kit of parts according to claim 1, wherein the size-classifier (12) is stacked on top of the separation unit (22) with the classifier floor (12f) replacing the separator lid (23) and wherein the driving coupling element (16c) of the classifier unit (12) also drives the rotation of the filter carousel (24) and wherein a clutch release mechanism allows the driving coupling element (16c) to drive independently or in combination two by two the carousel (14), the filter carousel (24) and the scraping unit (26).

11. The kit of parts according to claim 7, comprising a fluidization unit (32) for fluidising an adipose tissue and comprising,
a first chamber (31c) comprising a first aperture (31a), and a second chamber (32c) comprising a second aperture (32a), the first chamber being separated from the second chamber by a filtering member (32fi, i=1-4),
a first piston pump (301) fluidly coupled to the first chamber (31) via the first aperture (31a) and a second piston pump (302) fluidly coupled to the second chamber via the second aperture (32a), the first and second piston pumps (301, 302) being configured for being activated in opposition of phase, allowing forcing adipose tissue to flow back and forth from one of the first and second chamber to the other one of the first and second chambers through the filtering unit (32fi) to fluidify the adipose tissue.

12. The kit of parts according to claim 11, wherein the filtering member (32fi) comprises at least two filters (32f1, 32f2, 32f3, 32f4), a first filter (32f1) having openings larger than a second filter (32f2) and so on, and wherein the filtering member (32fi) is mounted such that each of the at least two filters can be brought successively into a position separating the first chamber (31c) from the second chamber (32c) and wherein the at least two filters (32f1, 32f2, 32f3, 32f4) are mounted on separate windows of a support frame (32s), wherein the support frame can be moved to bring each window loaded with one of the at least two filters (32f1, 32f2, 32f3, 32f4) in the position separating the first chamber (31c) from the second chamber (32c).

13. A liposuction assembly obtained by assembling the components of the kit-of parts of claim 6 coupled to one another by transfer tubes (6t).

14. The liposuction assembly according to claim 13, wherein
a transfer tube (6t) is coupled in fluid communication to the cannula outlet of the liposuction unit (2) and the filter inlet opening (22i),
a transfer tube (6t) is coupled in fluid communication to the retentate outlet opening of the retentate unit (22) and the classifier inlet opening (12i),
a rotation transmission cable (5m) is coupled to the liposuction unit and, optionally to the carousel of the size-classifier (22) and/or to the filter carousel of the filtering unit.

15. The liposuction assembly according to claim 13, wherein
a transfer tube (6t) is coupled in fluid communication to the cannula outlet of the liposuction unit (2) and the filter inlet opening (22i),
a transfer tube (6t) is coupled in fluid communication to the retentate outlet opening (22or) of the retentate unit (22) and the first piston pump (301),
a transfer tube (6t) is coupled in fluid communication to the second piston pump (302) to the classifier inlet opening (12i) of the size-classifier (12),
a rotation transmission cable (5m) is coupled to the liposuction unit,
wherein the liposuction assembly comprises first and second fluidisation valves (31v, 32v) located upstream and downstream of the fluidization unit (32) to allow the latter to be isolated from the rest of the assembly and to allow the flowing back-and-forth of adipose tissue enclosed between the first and second valves.

16. The size-classifier (12) according to claim 1, wherein the open end is sealed by a membrane (14m).

17. The size-classifier (12) according to claim 1, wherein the needle direct access to the compartments (14i) is through the membrane (14m).

18. The size-classifier (12) according to claim 2, wherein the first diameter is-between 400 and 800 μm.

19. The size-classifier (12) according to claim 2, wherein the (n−1)^{th} diameter is

20. The size-classifier (12) according to claim 3, wherein the angle (α) is between 45 and 120°.
